(19) United States
(12) Patent Application Publication
(10) Pub. No.: US 2017/0348218 A1
(43) Pub. Date: Dec. 7, 2017

(54) MICRONEEDLE PATCH CONTAINING HYALURONIC ACID FOR COSMETIC USE

(71) Applicant: Sanova Bioscience Inc, Torrance, CA (US)

(72) Inventors: Meichin CHEN, Tainan City (TW); Chiasui CHEN, Tainan City (TW); Bingrong LIU, Rancho Palos Verdes, CA (US); Zihyao LIN, Tainan City (TW)

(73) Assignee: Sanova Bioscience Inc, Torrance, CA (US)

(21) Appl. No.: 15/184,126

(22) Filed: Jun. 16, 2016

Related U.S. Application Data
(60) Provisional application No. 62/345,143, filed on Jun. 3, 2016.

(51) Int. Cl. A61K 8/73; A61Q 19/08

(52) U.S. Cl. CPC A61K 8/735; A61Q 19/08

(57) ABSTRACT

The disclosure provides microneedle patches for delivering hyaluronic acid (“HA”) to skin and methods of using the microneedle patches of the disclosure to maintain and/or improve the appearance of skin.

### 1. CROSS REFERENCE TO RELATED APPLICATIONS

The application claims the priority benefit of U.S. provisional application No. 62/345,143, filed Jun. 3, 2016, the contents of which are incorporated by reference in their entireties.

### 2. BACKGROUND

Hyaluronic acid (“HA”) is a naturally occurring biodegradable polymer composed of repeating disaccharides (β-1,4-D-glucuronic acid (known as uronic acid) and β-1,3-N-acetyl-D-glucosamide) and is a major component in the extracellular matrix of vertebrate tissues. HA based injectable dermal fillers have become the “gold standard” for treating wrinkles, hydrating skin, and increasing skin volume since their introduction two decades ago (Fakhari et al., 2014, Acta Biomater. 9(7): 7081-7092). Several HA based dermal fillers have been brought to market over the past two decades, including Restylane® (Galderma Laboratories, L.P.), Perlane® (Galderma Laboratories, L.P.), Hylaform® (Genzyme Corporation), Juvederm® (Allergan), and Surgiderm® (Allergan). However, injectable dermal fillers have several disadvantages including pain during and after injection, and potential side effects such as skin redness, swelling, tenderness, firmness, lumps/bumps, discoloration, and bruising (Juvederm® Ultra XC directions for use (www.allergan.com/miscellaneous-pages/allergan-pdf-files/juvederm_ultra_xc_dfu)).

HA containing microneedle patches can avoid at least some of the disadvantages associated with injectable HA based dermal fillers, e.g., injection pain. Several HA containing microneedle patches are commercially available, including Quanis Dermafiller™ (CosMED Pharmaceutical Co., Ltd.), HA Fill Patch™ (Navision), Acropass™ (Raphas Co. Ltd.), MESO Patch™ (Raphas Co. Ltd.), MicroCure™ (Nissha Printing Co., Ltd.), Royal Skin™ Hyaluronic Acid Micro Patch (Junmok International), Wellage™ Hyaluronic Acid Micro Needle Patch (Wellage), and Time Patch™ (Storyderm). However, the instructions for using these microneedle patches generally recommend that the patches be worn for at least 30 minutes and often recommend that the patches be worn overnight to allow sufficient time for the HA containing microneedles to dissolve in the skin (quanis.jp/en/lineup/dermafiller/; Matsunaga, 2015, Drug Delivery System 30-4:371-376; www.acropass.com/product/EyezoneCare.html; en.koreadepart.com/item/1452219176/wellagehyaluronicacidmicroneedlepatch).

Thus, new devices and methods for delivering HA to skin are needed that avoid the disadvantages of injectable HA containing dermal fillers and available HA containing microneedle patches.

### 3. SUMMARY

The present disclosure provides microneedle patches for delivering hyaluronic acid (“HA”) to skin and methods of using the microneedle patches of the disclosure to maintain and/or improve the appearance of skin. The microneedle patches of the disclosure comprise a plurality of HA containing microneedles immobilized on a substrate or backing. The shafts and tips of the microneedles are formulated of non-identical components that allow the shafts dissolve faster than the tips when applied to skin. The shafts can be formulated so that the shafts dissolve in a short amount of time after applying the microneedle patch to the skin (e.g., about 5-10 minutes or less), thereby separating the tips from the shafts and allowing the user to remove the backing from the skin shortly after application. The tips remaining in the skin after the shafts dissolve can slowly release HA into the skin even after the backing has been removed. Application of the microneedle patches of the disclosure to the skin can, for example, help to reduce the appearance of fine lines and wrinkles, increase the moisture content of the skin, and improve skin's elasticity. The microneedles can further comprise one or more optional cosmetic agents to provide additional cosmetic benefits beyond those provided by HA alone. The microneedle patches of the disclosure are described in detail in Section 5.3, and methods of using the microneedle patches for maintaining and/or improving the appearance of skin are described in detail in Section 5.4.

The present disclosure also provides kits comprising the microneedle patches of the disclosure. The kits can contain, for example, a plurality of microneedle patches, a microneedle patch applicator, a cleaning agent for cleaning the skin prior to application of a microneedle patch, a composition for application to the skin after the microneedle patch is removed, instructions for using the microneedle patches, or any combination thereof. Kits comprising the microneedle patches of the disclosure are described in Section 5.5.

### 4. BRIEF DESCRIPTION OF THE FIGURES

FIG. 1 illustrates an exemplary microneedle of the disclosure.

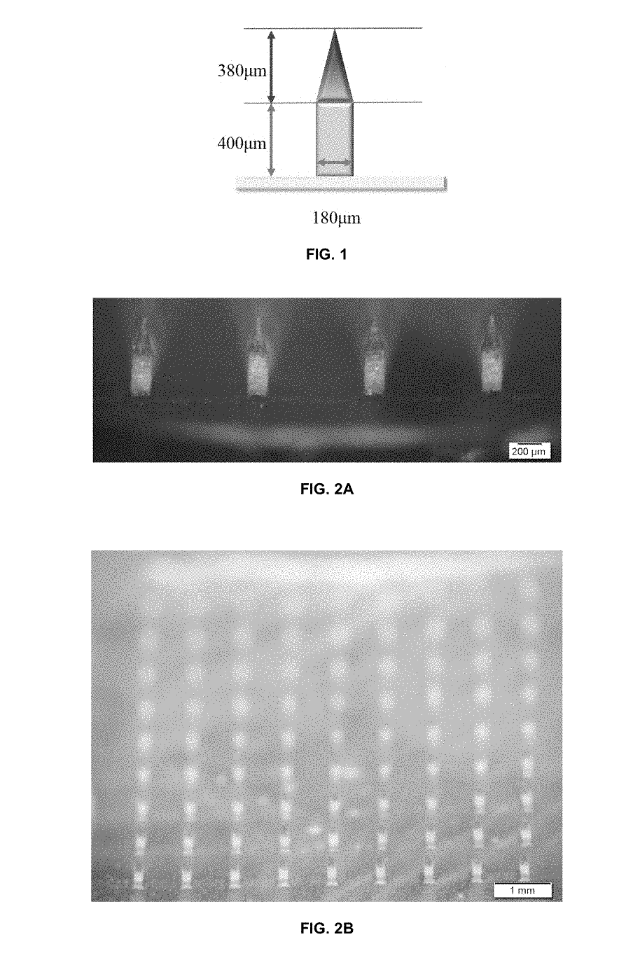

FIGS. 2A-2D are photographs of the microneedles of the microneedle patches described in Example 1 taken at different distances.

FIGS. 3A-3B are photographs of porcine skin following application of a microneedle patch as described in Example 1. FIG. 3A shows microneedle tips (labeled with blue dextran) remaining in the skin following removal of the patch. FIG. 3B shows sites of four microneedle insertions (denoted by arrows).

FIGS. 4A-4B are photographs of skin from Sprague-Dawley rats following application of a microneedle patch as described in Example 2. FIG. 4A shows microneedle tips (labeled with Rhodamine) remaining in the skin following removal of the patch. FIG. 4B shows sites of microneedle insertions. The top panel shows a section of skin to which the microneedle patch was applied. The middle panel shows the Rhodamine fluorescence from several tips that remain in the skin. The bottom panel is an overlay of the top and middle panels.

FIGS. 5A-5B are photographs of skin from Sprague-Dawley rats following application of a microneedle patch as described in Example 2. FIG. 5A shows microneedle tips (labeled with Rhodamine) remaining in the skin following removal of the patch. FIG. 5B shows sites of microneedle insertions. The top panel shows a section of skin to which the microneedle patch was applied. The middle panel shows the Rhodamine fluorescence from several tips that remain in the skin. The bottom panel is an overlay of the top and middle panels.

### 5. DETAILED DESCRIPTION

### 5.1. Overview

The present disclosure provides microneedle patches for delivering hyaluronic acid (“HA”) and, optionally, one or more cosmetic agents to skin and methods of using the microneedle patches to maintain and/or improve the appearance of skin by, for example, combatting effects associated with aging. A microneedle patch of the disclosure can be configured so that single or multiple applications of the microneedle patch result in maintenance or improvement of the appearance of skin to which the microneedle patch is applied.

The microneedle patches of the disclosure comprise a substrate (also referred to herein as a “backing”) having a plurality of HA containing microneedles immobilized thereon. The microneedles comprise shafts that extend outward from a surface of the substrate and terminate with tips having a composition that is different from the composition of the shafts such that the shafts dissolve faster than the tips when the microneedles are applied to skin. Advantageously, the different rates of dissolution can allow for a sustained release of the tip components (e.g., HA and one or more cosmetic agents, if present) into the skin even after the microneedle patch is removed from the skin. The microneedle patches of the disclosure are described in detail in Section 5.3 below, with the microneedle shafts being described in detail in Section 5.3.1, the microneedle tips described in detail in Section 5.3.2, the microneedle patch substrate described in detail in Section 5.3.3, and methods for making the microneedle patches described in Section 5.3.4. Cosmetic uses of the microneedle patches of the disclosure are described in Section 5.4. Kits containing the microneedle patches of the disclosure are described in Section 5.5.

### 5.2. Definitions

Average molecular weight refers to weight average molecular weight as determined by size exclusion chromatography, e.g., multi-angle laser light scattering-size exclusion chromatography (MALLS-SEC).

A cosmetic agent is a compound or composition intended to improve the appearance of skin.

Unless required otherwise by context, the term hyaluronic acid (“HA”) includes hyaluronic acid, salts thereof, (e.g., sodium hyaluronate, potassium hyaluronate, magnesium hyaluronate, calcium hyaluronate), and combinations of any of the foregoing.

### 5.3. Microneedle Patches

The microneedle patches of the disclosure contain a plurality of HA containing microneedles immobilized on a substrate. The individual microneedles comprise a shaft and an HA containing tip. The microneedles can further comprise one or more cosmetic agents. The microneedles are oriented on the substrate so that the microneedle shafts extend outward from the substrate and terminate with the microneedle tips.

The microneedles can be crystalline and can have a low moisture content, e.g., 0% to 10% moisture, 0% to 5% moisture, 1% to 10% moisture, 1% to 5% moisture, less than 10% moisture, less than 5% moisture, or less than 1% moisture.

The distance between each of the individual microneedles on the substrate can be selected to ensure efficient penetration of the skin to which the microneedle patch is intended to be applied so that the tips comprising HA and one or more cosmetic agents, if present, can be delivered to the skin. For example, the microneedles can be separated from each other on the substrate by a distance ranging from 50 μm to 2000 μm, 100 μm to 1500 μm, 100 μm to 1000 μm, 100 μm to 500 μm, 500 μm to 1000 μm 500 μm to 1500 μm, 500 μm to 2000 μm, 1000 μm to 1500 μm, 1000 μm to 2000 μm, 1500 μm to 2000 μm, or a distance in a range bounded by any two of the foregoing values.

The microneedles can be separated from each other on the substrate at regular intervals (e.g., in regularly spaced rows and/or columns), although microneedle patches having microneedles spaced at irregular intervals are also within the scope of the disclosure.

In some embodiments, the microneedle patch comprises multiple rows of microneedles in which the distance between microneedles is the same in all of the rows. In other embodiments, the microneedle patch comprises multiple rows of microneedles in which the distance between microneedles is not the same in all of the rows (e.g., the microneedle patch can comprise one or more rows in which the distance between the microneedles is distance A and one or more rows in which the distance between the microneedles is a different distance B).

The total number of microneedles on a microneedle patch can vary based upon the distance between the microneedles and the size of the patch. In some embodiments, the microneedle patch can comprise 10 to 20,000 microneedles, 100 to 10,000 microneedles, 100 to 5,000 microneedles, 1000 to 5000 microneedles, 1000 to 2500 microneedles or a range bounded by any two of the foregoing values. In some instances, it may be more convenient to refer to the number of microneedles per surface area of substrate rather than the total number of microneedles on a microneedle patch. In some embodiments, the number of microneedles per square centimeter ranges from 25 to 500, 25 to 200, 50 to 100, 75 to 100, 100 to 200, 200 to 400, or a range bounded by any two of the foregoing values.

The size and shape of the microneedle patch can be, for example, rectangular (with or without rounded edges), circular, oval, elliptical, or kidney shaped. Different size and shape microneedle patches can be made for application to different skin surfaces. For example, a microneedle patch for application around the eyes can be kidney shaped and sized to match the area surrounding the eye where crow's feet wrinkles typically form, and a microneedle patch for application to a forehead may be a rectangular shape. In some embodiments, the microneedle patch is about 1 to 20 cm long and about 1 to 10 cm wide, about 1 to 10 cm long and about 1 to 10 cm wide, about 2 to about 6 cm long and about 1 to 6 cm wide, or about 3 to about 6 cm long and about 2 to 6 cm wide.

5.3.1. Microneedle Shafts

The microneedle shafts are formulated such that the shafts can be dissolved by fluid present in or on skin (e.g., skin interstitial fluid, moisture present on the skin surface, or water or saline added to the skin after application of the microneedle patch) more readily than the microneedle tips. Thus, following application of the patch to skin and dissolution of the shafts, the microneedle patch can be removed from the skin while leaving behind a majority of the microneedle tips (e.g., more than 50%, more than 60%, more than 70%, more than 80%, more than 90%, more than 90%, more than 95%, more than 96%, more than 97%, more than 98%, more than 99% or 100% of the tips).

The microneedle shafts preferably comprise HA and, optionally, one or more cosmetic agents. The HA in the shafts, when present, preferably has an average molecular weight that is lower than the average molecular weight of the HA in the tips, although the average molecular weight of the HA in the shafts can be the same or greater than the average molecular weight of the HA in the tips so long as the compositions of the shafts and tips are selected so that the shafts dissolve faster than the tips (e.g., by including a fast dissolving polymer in the shafts and/or a slow dissolving polymer in the tips) when the microneedle patch is applied to skin. The average molecular weight of the HA in the shafts can range, for example, from about 5 kDa to about 500 kDa, about 5 kDa to about 100 kDa, about 5 kDa to about 50 kDa, about 5 kDa to about 25 kDa, about 5 kDa to about 10 kDa, or any range bounded by any two of the foregoing values. In an embodiment, the HA in the shafts has an average molecular weight of about 7 kDa.

The HA in the shafts can be a single population of HA (e.g., a population exhibiting a single SEC peak) or a mixture of multiple HA populations that each have a different average molecular weight (e.g., HA having an average molecular weight of 7 kDa and HA having an average molecular weight of 25 kDa).

The shafts can include components that provide the shafts with the desired characteristics, e.g., desired mechanical strength, flexibility, and dissolution profile. Materials suitable for inclusion in the shafts include water-soluble and/or water-swellable polymers. In certain embodiments, the shafts comprise HA and one or more additional polymers. While not being limited by theory, it is believed that shafts comprising HA and one or more polymers in addition to HA can provide the microneedle patches with increased strength and render them mechanically more robust as compared to shafts containing only HA, while at the same time providing a suitable dissolution rate. Thus, the components of the shafts can be selected so that the force required to insert the microneedles into skin is less than the force required to fracture the shafts.

In some embodiments, the microneedles do not fracture when a pressure of insertion of less than about 10 N/cm², less than about 9.5 N/cm², less than about 9 N/cm², less than about 8.5 N/cm², less than about 8 N/cm², less than about 7.5 N/cm², less than about 7 N/cm², less than about 6.5 N/cm², less than about 6 N/cm², less than about 5.5 N/cm², less than about 5.0 N/cm², less than about 4.5 N/cm², less than about 4 N/cm², or less than about 3.5 N/cm² is exerted on the microneedles along their length. In some embodiments, the microneedles can be configured to not break upon application of a defined force, such as about or no more than about 50, 45, 40, 35, 30, 25, 20, or 15 N per patch for a defined time period, such as about 30 seconds, about 45 seconds, about 60 seconds, about 1 minute, about 2 minutes, or about 3 minutes, about 4 minutes, or about 5 minutes, for example.

Exemplary polymers that can be included in the shafts include gamma-polyglutamic acid (“γ-PGA”), polyvinylpyrrolidone (“PVP”), poly(vinyl alcohol) (“PVA”), starch, gelatin, collagen, and combinations thereof. In some embodiments, the shafts comprise HA and one, two, three, four, five, or all of PVP, PVA, γ-PGA, starch, gelatin, and collagen. In an embodiment, the shafts comprise HA and γ-PGA. In another embodiment, the shafts comprise HA and PVA. In yet another embodiment, the shafts comprise HA, γ-PGA, and PVA. In yet another embodiment, the shafts comprise γ-PGA, PVA, or a combination of γ-PGA and PVA, but do not comprise HA.

The average molecular weight of a polymer other than HA included in the shafts (e.g., γ-PGA and/or PVA) can range, for example, from about 5 kDa to about 800 kDa, about 100 kDa to about 800 kDa, about 100 kDa to about 400 kDa, about 100 kDa to about 150 kDa, about 5 kDa to about 400 kDa, 5 kDa to about 200 kDa, about 5 kDa to about 150 kDa, about 5 kDa to about 100 kDa, or a range bounded by any two of the foregoing values. In an exemplary embodiment, the shafts comprise γ-PGA having an average molecular weight ranging from about 100 kDa to about 800 kDa and/or comprise PVA having an average molecular weight ranging from about 100 kDa to about 150 kDa.

In some embodiments, the one or more polymers other than HA can make up about 1% to about 100% of the shafts by weight, about 1% to about 80% of the shafts by weight, about 20% to 80% of the shafts by weight, about 40% to about 80% of the shafts by weight, about 60% to about 80% of the shafts by weight, or any range between any of the foregoing values, with the balance being HA. In an exemplary embodiment, the shafts comprise HA and one or more polymers in a weight ratio of about 1:2.

In some embodiments, the composition of the shafts is selected so that the shafts dissolve following insertion of the microneedles into skin in within about 1 minute to about 60 minutes, about 1 to about 30 minutes, about 1 minute to about 15 minutes, about 1 minute to about 10 minutes, about 1 minute to about 5 minutes, about 2 minutes to 10 minutes, about 2 minutes to about 5 minutes, about 3 minutes to about 10 minutes, about 3 minutes to about 5 minutes, or a range bounded by any two of the foregoing values. In exemplary embodiments, the shafts dissolve in about 1 minute or less, about 2 minutes or less, about 3 minutes or less, about 4 minutes or less, about 5 minutes or less, about 6 minutes or less, about 7 minutes or less, about 8 minutes or less, about 9 minutes or less, about 10 minutes or less, or about 10 minutes or less following insertion of the microneedles into skin. The dissolution time for the shafts can be lengthened or shortened by, for example, varying the relative amounts and/or molecular weights of the shaft components.

The shafts can optionally include one or more cosmetic agents. A cosmetic agent can be distributed throughout the shafts (e.g., by including the cosmetic agent in a solution or gel used to make the shafts) or can be coated on the exterior of the shafts (e.g., by dipping microneedle shafts in a solution comprising the cosmetic agent or spraying a solution comprising the cosmetic agent on the shafts). Cosmetic agents that can be used in the microneedle patches include whitening agents, antiwrinkle agents (such as antioxidants), and vitamins. Exemplary whitening agents include ascorbic acid disodium phosphate, ascorbic acid glucoside, α(β)-arbutin, ascorbyl palmitate, kojic acid, resorcinol, tranexamic acid, licorice extract, and retinoin. Exemplary antiwrinkle agents include hydrolyzed collagen, ectoin, retinol, tretinoin, retinol acetate, vitamin A palmitate, magnesium ascorbyl phosphate, trisodium ascorbyl palmitate phosphate, resveratrol, adenosine, epidermal growth factor, peptides, and caffeine. Exemplary vitamins include Vitamin C, vitamin D₂, vitamin D₃, vitamin K, and derivatives of the foregoing.

The shafts can be any appropriate cross-section, such as triangular, rectangular, circular, oval, or elliptical, for example, and/or take the form of cylinders or prisms (e.g., having a base with 3, 4, 5, 6, 7, 8, or more than 8 edges). In some embodiments, the shafts have a circular cross-section. In other embodiments, the shafts have a rectangular or square cross-section.

The height of the shafts can be selected in conjunction with the height of the tips so that the microneedles penetrate into the skin at an appropriate depth when the microneedle patch is applied to a subject's skin. The height of the shafts is preferably selected in order to prevent over-penetration of the tips which may cause pain or harm to a subject. In some embodiments, the height of the shafts can range from 200 μm to 1000 μm, 200 μm to 600 μm, 300 μm to 500 μm, or any range bounded by any two of the foregoing values. In a particular embodiment, the height of the shafts is about 400 μm. In some embodiments, the height of the microneedles is about 400 μm to about 1200 μm, about 500 μm to about 1000 μm, or about 600 μm to about 800 μm.

The width of the shafts can range from about 100 μm to about 500 μm, about 100 μm to about 250 μm, about 150 μm to about 200 μm, or any range bounded by any two of the foregoing values. In preferred embodiments, the shafts have a uniform width throughout their length. In an embodiment, the width of the shafts is about 180 μm.

5.3.2. Microneedle Tips

Microneedle tips suitable for inclusion in the microneedle patches of the disclosure comprise HA and, optionally, one or more cosmetic agents. In an embodiment, the tips consist essentially of HA. In another embodiment, the tips consist essentially of HA and one or more cosmetic agents.

The composition of the tips is selected so that the tips dissolve more slowly than the shafts when the microneedles are inserted into skin. In some embodiments, the tips dissolve following insertion of the microneedles into skin in within about 5 to about 60 minutes, about 5 to 30 minutes, about 5 to 15 minutes, about 5 to 10 minutes, or any range bounded by any two of the foregoing values.

In exemplary embodiments, the tips dissolve in about 5 minutes, about 10 minutes about 15 minutes, about 20 minutes, about 25 minutes or about 30 minutes following insertion into skin. The dissolution time for the tips can be lengthened or shortened by, for example, varying the relative amounts and/or molecular weights of the tip components, e.g., the molecular weight of the HA.

The HA in the tip preferably has an average molecular weight that is higher than the average molecular weight of the HA in the shafts, if present, although the average molecular weight of the HA in the tips can be the same or less than the average molecular weight of the HA in the shafts so long as the compositions of the shafts and tips are selected so that the shafts dissolve faster than the tips (e.g., by including a fast dissolving polymer in the shafts and/or a slow dissolving polymer in the tips) when the microneedle patch is applied to skin. The average molecular weight of the HA in the tips can range, for example, from about 5 kDa to about 4 MDa, about 100 kdDa to about 1 MDa, about 100 kDa to about 800 kDa, about 100 kDa to about 600 kDa, about 100 kDa to about 500 kDa, about 200 kDa to about 500 kDa, or any range bounded by any two of the foregoing values. In an embodiment, the HA in the tips has an average molecular weight from about 200 kDa to about 500 kDa. The HA in the tips can be made from a single population of HA (e.g., a population exhibiting a single SEC peak) or a mixture of multiple HA populations that each have a different average molecular weight (e.g., HA having an average molecular weight of 200 kDa and HA having an average molecular weight of 500 kDa). The HA in the tip can be chemically crosslinked to reduce its rate of degradation in the skin, for example, with 1,4-butanediol diglycidal ether or divinyl sulfone.

The tips can include components in addition to HA to provide tips having desired characteristics, e.g., desired mechanical strength, flexibility, and dissolution profile. Materials suitable for inclusion in the tips in addition to HA include water-soluble and/or water-swellable polymers. Exemplary polymers that can be included in the tips include polyvinylpyrrolidone (“PVP”), poly(vinyl alcohol) (“PVA”), γ-poly-glutamic acid (“γ-PGA”), starch, gelatin, collagen, chitosan, chitin, silk, carboxymethyl cellulose, chondroitin sulfate, polycaprolactone, poly(methyl vinyl ether-maleic anhydride), polyacrylic acid, poly(2-hydroxyethyl methacrylate), poly(N,N-dimethyl acrylamide), maltose, polylactic acid, polyglycolic acid, poly(lactic-co-glycolic acid), glycogen, dextrin, dextran, hydroxypropyl methylcellulose, alginic acid, pullulan, carboxyvinyl polymer, salts thereof, and combinations thereof. In one embodiment, the tips include one or more of chitosan, chitin, silk, carboxymethyl cellulose, chondroitin, collagen, gelatin, polycaprolactone, poly(methyl vinyl ether-maleic anhydride), polyacrylic acid, 2-hydroxyethyl methacrylate, N,N-dimetyl acrylamide, maltose, polylactic acid, polyglycolic acid, and poly(lactic-co-glycolic acid).

The microneedle tips can be formulated to absorb skin interstitial fluid prior to their degradation such that the tips increase in volume (e.g., in order to provide an improved aesthetic appearance to the skin). In some embodiments, the tips can increase in weight (e.g., by the absorption of interstitial fluid) following insertion into skin by about 10% to about 1000%, about 10% to about 100%, about 100% to about 500%, or any range bounded by any two of the foregoing values.

The tips can optionally include one or more cosmetic agents, e.g., one or more of the cosmetic agents described in Section 5.3.2. A cosmetic agent can be distributed throughout the tips (e.g., by including the cosmetic agent in a solution or gel used to make the tips) or can be coated on the exterior of the tips (e.g., by dipping the tips of a microneedle patch of the disclosure in a solution comprising the cosmetic agent or spraying the tips with a solution comprising the cosmetic agent).

The tips can further comprise a material capable of melting after exposure to radiation. Radiation that can be used to melt the tips includes electromagnetic radiation such as near-infrared light, infrared light, or microwave. Exemplary materials capable of melting after exposure to radiation include indocyanine green, polycaprolactone (PCL), gelatin, methylcellulose, or polyethylene oxide (PEO), and nanoparticles. The nanoparticles can be metal nanoparticles, for example, gold nanoparticles, gold nanorods, gold and silver nanoballs, or germanium nanoparticles. The nanoparticles can also be single-walled carbon nanotubes, or lanthanum hexaboride nanoparticles. When radiation is applied to the tips, the material capable of melting absorbs the radiation and converts the radiation into heat, thereby melting the tips and releasing the components of the tip into the skin.

The shape of the tips can be any shape which can penetrate skin. For example, the tips can be cone shaped or square pyramidal shaped. In some embodiments, the base of the tips, which join the tips to the shafts, has the same shape as the cross-sectional shape of the shafts.

The height of the tips can be selected in conjunction with the height of the shafts so that the tips penetrate into the skin at an appropriate depth when the microneedle patch is applied to a subject's skin in order to prevent over-penetration which may cause pain or harm to the subject. In some embodiments, the height of the tips can range from 200 μm to 1000 μm, 200 μm to 600 μm, 300 μm to 460 μm, or any range bounded by any two of the foregoing values. In a particular embodiment, the height of the tips is about 380 μm±20 μm.

The width of the tips at their base (i.e., where the tips contact the shafts) can range from about 100 μm to about 500 μm, about 100 μm to about 250 μm, about 150 μm to about 200 μm, or any range bounded by any two of the foregoing values. In an embodiment, the width of the tips at their base is about 180 μm±20 μm. The width of the tips at their base can be, but is not necessarily, the same as the width of the shafts to which they are attached. For example, the width of the tips at their base can be wider than the width of the shafts.

The width of the tips at their top can range, for example, from about less than 1 μm to 20 μm, from about 1 μm to about 20 μm, from about 1 μm to about 15 μm, from about 1 μm to about 10 μm, from about 1 μm to about 5 μm, from about 5 μm to about 20 μm, from about 5 μm to about 15 μm, from about 5 μm to about 10 μm, from about 10 μm to about 20 μm, from about 10 μm to about 15 μm, from about 15 μm to about 20 μm, or any range bounded by any two of the foregoing values.

5.3.3. Microneedle Substrates

The microneedle patches of the disclosure comprise a substrate to which the microneedles are attached. The substrate can be made of the same material as the shafts or can be different. Thus, the materials identified in Section 5.3.1 can be used to make a substrate for the microneedle patches of the disclosure. When the substrate comprises the same material as the shafts, the shafts and substrate can be integrally formed. The substrate can be formulated so that the microneedle patch is flexible and/or stretchable. In some embodiments, the substrate is made with materials having a high mechanical strength, such as PVP, PVA, γ-PGA, HA, starch, gelatin, or their combinations. In a preferred embodiment, the substrate comprises γ-PGA.

Other materials that can be used as a substrate include, but are not limited to, single-sided tape, double-sided tape, and fabric, each of which can be air-permeable

The thickness of the substrate can be, for example, about 100 μm to about 5000 μm, about 100 μm to about 2500 μm, or about 100 μm to about 1000 μm.

Multiple types of substrate materials may be used, for example to provide protection to the patch during transport and/or to aid in application of the patch. In some embodiments, the substrate comprises a polymer, e.g., γ-PGA, and double-sided tape or fabric, wherein the polymer is situated between the double-sided tape or fabric and the microneedle shafts.

5.3.4. Methods of Making Microneedle Patches

Methods for making microneedle patches are known in the art and can be used to make the microneedle patches of the disclosure (see, e.g., US 2014/0005606 and US 2015/0018755, the contents of which are incorporated herein by reference in their entireties).

In some embodiments, the microneedle patches are formed by casting. The microneedle patches can be formed by a multi-stage casting process wherein the tips, shafts, and substrate are formed in two or more stages. For example, a mixture comprising the tip component(s) can be added to a polydimethylsiloxane (“PDMS”) microneedle mold and centrifuged to fill the tips of the mold. The mold can then be dried, thereby forming the microneedle tips in the mold. Separately, a mixture comprising the shaft component(s) can be added to a second PDMS mold and centrifuged to fill the cavities of the mold. The second mold can then be dried, thereby forming the microneedle shafts in the second mold. A solution comprising the substrate component(s) can then be added on top of the shafts in the second mold. The second mold can then be dried again, thereby forming the substrate attached to the microneedle shafts. The substrate having the attached shafts can then be removed from the second mold, dipped in an adhesive solution (e.g., a solution comprising one or more polymers that are present in the shafts and/or tips), aligned with the cavities of the first mold having the tips located therein, and then gently pressed down so that the shafts stick to the tips. The complete microneedle patch can then be removed from the first mold.

Optional cosmetic agents can be incorporated into the microneedles by including the cosmetic agents in the mixture comprising the shaft components and/or the mixture comprising the tip components prior to forming the shafts and/or tips. Alternatively, the cosmetic agents can be added to the microneedle patch after formation by soaking the microneedle shafts and/or microneedle tips in a solution comprising the cosmetic agents causing the shafts and/or tips to swell with the solution comprising the cosmetic agent, and thereafter drying the shafts and/or tips. Alternatively, a pre-formed formed microneedle patch can be dipped into a solution comprising the cosmetic agents or sprayed with a solution comprising the cosmetic agents so that the cosmetic agents are deposited on the surface of the shafts and/or tips.

### 5.4. Methods of Using the Microneedle Patches

The disclosure provides methods of using the microneedle patches described herein to deliver HA to the skin of a subject. The subject is preferably a human, e.g., an adult over the age of 20, over the age of 30, over the age of 40, over the age of 50, or over the age of 60. In some embodiments, the subject is female.

HA is useful for improving the appearance of skin imperfections caused by aging (e.g., fine lines, wrinkles, and volume loss) and reducing the appearance of scars (e.g., stretch marks and acne scars). In particular, HA is useful for moisturizing skin, reducing the appearance of fine lines, reducing the appearance of wrinkles, refining pores, and restoring and/or maintaining skin volume, texture, elasticity, and strength. Accordingly, the microneedle patches of the disclosure can be used to maintain and/or improve the appearance of skin by delivering HA to the skin.

In one aspect, the methods comprise applying a microneedle to an area of skin, such as an area of the face. Exemplary areas of skin to which the microneedle patch can be applied include the skin surrounding the mouth, skin surrounding the eyes, the forehead, or any area on the face or body which has a fine line, wrinkle, or scar.

The microneedle patch can be removed following dissolution of the microneedle shafts in the skin or on the skin's surface. In exemplary embodiments, the microneedle patch can be removed from the skin after about 1 minute to about 30 minutes, about 1 minute to about 15 minutes, about 1 minute to about 10 minutes, about 1 minute to about 5 minutes, about 2 minutes to about 15 minutes, about 2 minutes to about 10 minutes, about 2 minutes to about 5 minutes, about 3 minutes to about 15 minutes, about 3 minutes to about 10 minutes, about 3 minutes to about 5 minutes, about 4 minutes to about 15 minutes, about 4 minutes to about 10 minutes, about 4 minutes to about 5 minutes, about 5 minutes to about 15 minutes, about 5 minutes to about 10 minutes, about 3 minutes, about 4 minutes, about 5 minutes, about 6 minutes, about 7 minutes, about 8 minutes, about 9 minutes, or about 10 minutes.

The methods can comprise a single application or multiple applications of a microneedle patch of the disclosure to an area of skin. It should be understood that the phrases “multiple applications,” “repeated applications,” “applying the microneedle patch multiple times” or the like means that a new patch is used for each application. Multiple applications can include application of the microneedle patch at regular intervals (e.g., daily, weekly, twice a week, three times a week, more than three times a week, monthly, semi-annually or annually) or at irregular intervals. The multiple applications can be continued indefinitely (e.g., to maintain the appearance of skin or to slow the development of signs of aging), continued until a desired result is achieved (e.g., a reduction in the appearance of fine lines, a wrinkle, or scar), continued for a predetermined period of time (e.g., about 1 week, about 4 weeks, about 8 weeks, about 12 weeks, about 1 month, about 1 to 6 months, about 6 months to about 1 year, etc.), or continued for at least a minimum amount of time (e.g., at least 1 week, at least 4 weeks, at least 8 weeks, at least 12 weeks, at least 1 month, at least 6 months, at least 1 year, etc.).

In some embodiments, single or repeated applications of the microneedle patches of the disclosure can improve the appearance of skin imperfections caused by aging. For example, single or repeated applications of the microneedle patch may reduce the appearance of fine lines and/or wrinkles (for example, nasolabial folds and/or crow's feet and/or forehead wrinkles), increase the moisture level of the skin, refine pores (i.e., reduce the appearance of pores), increase skin volume, improve skin texture (e.g., smooth skin texture), improve skin elasticity, improve skin strength, or any combination thereof.

In some embodiments, single or repeated applications of the microneedle patches of the disclosure can reduce the appearance of stretch marks or acne scars to which the microneedle patch is applied.

A microneedle patch can be applied by hand by pressing the patch into the skin (e.g., for about 10 seconds to about 3 minutes, about 30 seconds to about 3 minutes, about 1 minute, about 2 minutes, or about 3 minutes) or can be applied with a mechanical microneedle patch applicator applying a predetermined force to help ensure that an optimal and/or uniform amount of force is applied to the patch during application (e.g., as described in WO 2008/091602 and WO 2014/153447, the contents of which are incorporated herein by reference in their entireties). An additional device may be used to reduce the elasticity of skin by stretching, pinching or pulling the surface of the skin so as to facilitate insertion of the microneedles. Alternatively, the skin can optionally be stretched by hand.

Water or a pharmaceutically acceptable solution (e.g., water, saline or phosphate buffered saline) can be applied to the skin before or after the microneedle patch is applied to the skin to accelerate the dissolution of the shafts and separation of the tips from the shafts.

The methods of using the microneedle patches described herein can further comprise application of radiation to the site of application if the tips contain a material capable of melting after exposure to radiation, for example, as described in Section 5.3.2. Radiation that can be used to melt the tips includes electromagnetic radiation such as near-infrared light, infrared light, or microwave radiation.

### 5.5. Kits

The disclosure further provides kits comprising one or more microneedle patches of the disclosure. A kit can comprise one or more microneedle patches (e.g., 1 to 100, 2 to 60, or 5 to 30 microneedle patches) together with a container for holding the one or more microneedle patches (e.g., a box, a pouch, or a wrapper), an agent for cleaning an application site, a microneedle applicator device (e.g., as described in WO 2008/091602 or WO 2014/153447), a composition for application to the skin after the microneedle patch is removed, instructions for using the microneedle patches according to a method described in Section 5.4, or a combination thereof. Exemplary agents for cleaning an application site are known in the art and include single-use wipes and cleansers. Single-use wipes and/or cleansers can contain a soap, a detergent, and/or an antiseptic such as an alcohol (e.g., ethanol or isopropranol), hydrogen peroxide, or iodine. Exemplary compositions that can be included in the kits for application to the skin after removal of the microneedle patch include lotions, creams, and ointments containing, for example, moisturizers, anti-inflammatory agents, and/or counterirritants.

### 6. EXAMPLES

### 6.1. Example 1: Delivering HA Containing Microneedle Tips to Porcine Skin

Microneedle patches having the composition and dimensions shown in Table 1 were evaluated for their ability to deliver HA containing tips to porcine skin.

TABLE 1
microneedle patch composition

| Patch component | Composition | Dimensions |
|---|---|---|
| Microneedle tips | 200-500 kDa HA | Height: 380 μm |
| | | Width at base: 180 μm |
| Microneedle shafts | 7 kDa HA and 100 kDa γ- | Height: 400 μm |
| | PGA in a 1:2 weight ratio | Width: 180 μm |
| Substrate | 100 kDa γ-PGA | |

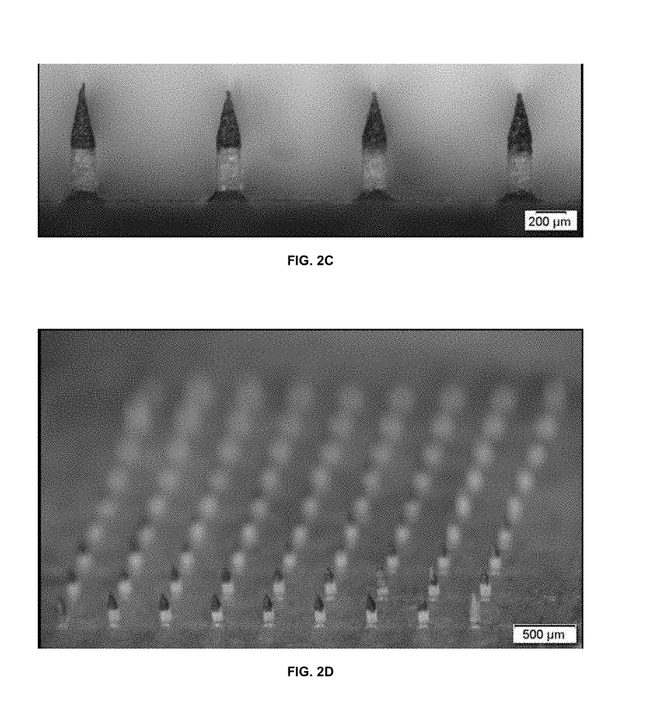

The microneedle patches used in this example contained 81 microneedles in a 1 cm² area. The microneedle tips were labeled with blue dextran to aid in visualization (see FIGS. 2A-2D).

Each microneedle patch was applied by hand to an ex vivo section of porcine skin by placing the patch on the skin and then pressing the patch from its center to its sides. Pressure was maintained on the patch for 3 minutes by holding a finger on the patch. After the 3 minutes, each patch was removed from the skin, embedded in an OCT gel consisting of polyethylene glycol and polyvinyl alcohol, placed in an aluminum-foil container, frozen with liquid nitrogen, and stored at −20° C. Following storage, the gels were sliced using a freezing microtome and analyzed to determine the depth of skin puncture and the spread of HA within the skin.

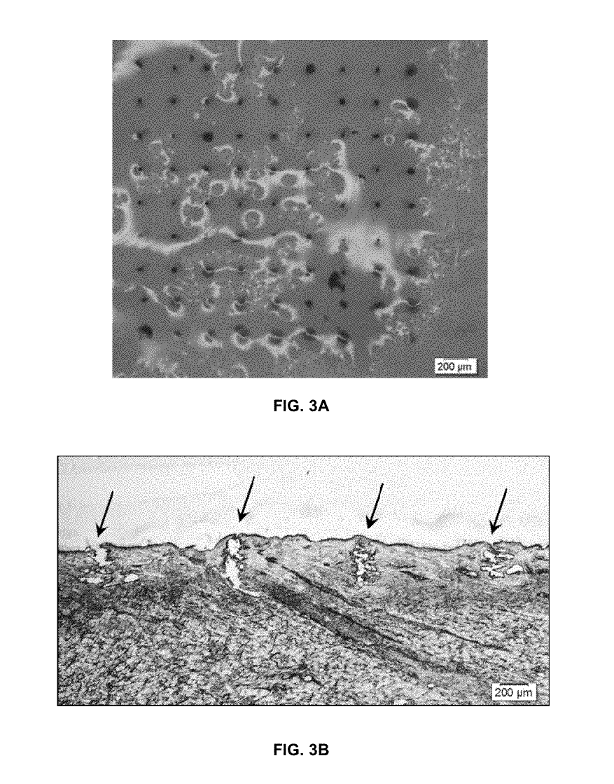

An exemplary skin section is shown in FIG. 3A. The distribution of dextran blue indicates that the success rate of skin puncture by the microneedles was 100%. The average depth of the microneedle punctures was 315±75 μm (see FIG. 3B).

### 6.2. Example 2: Delivering HA Containing Microneedle Tips to Skin of Live Rats

Microneedle patches having the composition and dimensions shown in Table 1 were evaluated for their ability to deliver HA containing tips to the skin of live Sprague-Dawley rats about 20 weeks old. The microneedle patches used in this example contained 81 microneedles in a 1 cm² area and the microneedle tips were labeled with Rhodamine to aid in visualization.

Each microneedle patch was applied by hand to a section of a live rat's skin by placing the patch on the skin and then pressing the patch from its center to its sides. Pressure was maintained on the patch for 3 minutes by holding a finger on the patch. After the 3 minutes, each patch was removed from the skin, the area of patch application was marked with a marking pen, and the marked skin sections were removed using blunt end dissecting scissors after sacrificing the rats by CO₂ gas inhalation. The skins were then embedded in an OCT gel consisting of polyethylene glycol and polyvinyl alcohol, placed in an aluminum-foil container, frozen with liquid nitrogen, and stored at −20° C. Following storage, the gels were sliced using a freezing microtome, and analyzed to determine the depth of skin puncture and the spread of HA within the skin.

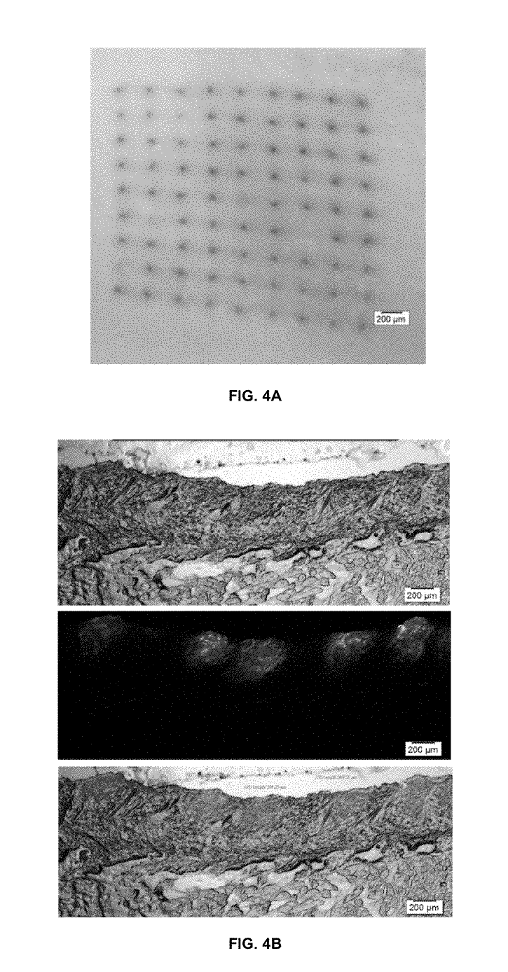

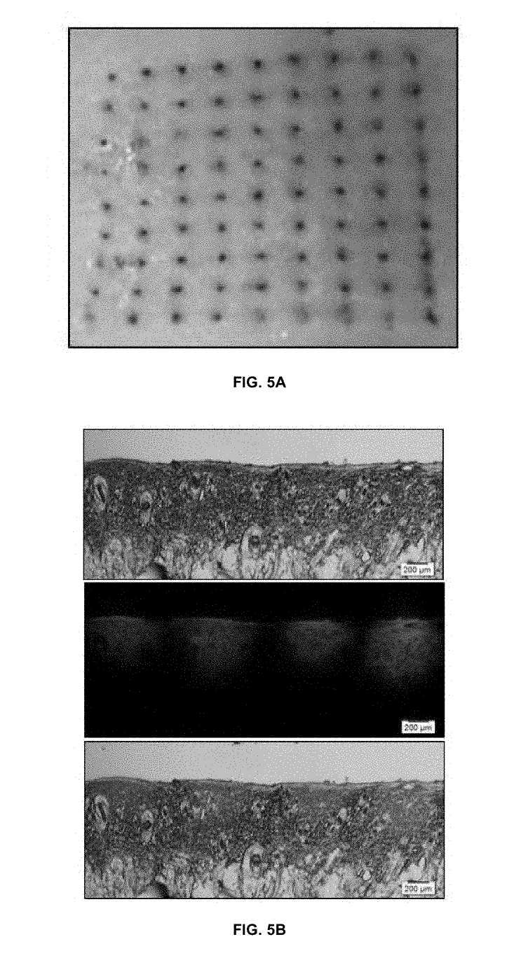

An exemplary skin section is shown in FIG. 4A. The distribution of Rhodamine indicates that the success rate of skin puncture by the microneedles was approximately 90-95%. The average depth of the microneedle punctures was 316±47 μm (see FIG. 4B). A second exemplary skin section in shown in FIG. 5A. The average depth of the microneedle punctures in the second skin section was 438.1±68.9 μm (see FIG. 5B).

The results of Examples 1 and 2 confirm that microneedle patches according to the disclosure can successfully deliver microneedle tips containing HA into skin.

### 7. SPECIFIC EMBODIMENTS

The present disclosure is exemplified by the specific embodiments below.

1. A microneedle patch for delivering hyaluronic acid (“HA”) to skin, comprising a substrate and a plurality of microneedles immobilized on the substrate, wherein the microneedles comprise:
(a) water dissolvable shafts; and
(b) tips comprising HA;
   wherein when the patch is applied to skin the shafts dissolve faster than the tips dissolve.

2. The microneedle patch of embodiment 1, wherein when the patch is applied to skin the shafts dissolve in about 1 minute to about 15 minutes, about 1 minute to about 10 minutes, about 1 minute to about 5 minutes, about 2 minutes to 10 minutes, about 2 minutes to about 5 minutes, about 3 minutes to about 10 minutes, or about 3 minutes to about 5 minutes.

3. The microneedle patch of embodiment 1 or embodiment 2, wherein when the patch is applied to skin the shafts dissolve in about 1 minute, about 2 minutes, about 3 minutes, about 4 minutes, about 5 minutes, about 6 minutes, about 7 minutes, about 8 minutes, about 9 minutes, or about 10 minutes.

4. The microneedle patch of any one of embodiments 1 to 3, wherein the shafts comprise HA.

5. The microneedle patch of embodiment 4, wherein the HA in the tips has an average molecular weight that is higher than the average molecular weight of the HA in the shafts.

6. The microneedle patch of any one of embodiments 1 to 5, wherein the average molecular weight of the HA in the tips ranges from about 5 kDa to about 4 MDa, about 100 kdDa to about 1 MDa, about 100 kDa to about 800 kDa, about 100 kDa to about 600 kDa, about 100 kDa to about 500 kDa, or about 200 kDa to about 500 kDa.

7. The microneedle patch of embodiment 6, wherein the average molecular weight of the HA in the tips ranges from about 200 to about 500 kDa.

8. The microneedle patch of any one of embodiments 4 to 7, wherein the average molecular weight of the HA in the shafts ranges from about 5 kDa to about 100 kDa, about 5 kDa to about 50 kDa, about 5 kDa to about 25 kDa, or about 5 kDa to about 10 kDa.

9. The microneedle patch of embodiment 8, in which the average molecular weight of HA in the shafts is about 7 kDa.

10. The microneedle patch of any one of embodiments 1 to 9, wherein the shafts comprise a polymer that is not HA.

11. The microneedle patch of embodiment 10, wherein the polymer comprises gamma-polyglutamic acid (“γ-PGA”), polyvinylpyrrolidone (“PVP”), poly(vinyl alcohol) (“PVA”), starch, gelatin, collagen, or a combination thereof.

12. The microneedle patch of embodiment 11, wherein the shafts comprise γ-PGA.

13. The microneedle patch of embodiment 12, in which the shafts comprise HA and γ-PGA and the weight ratio of HA to γ-PGA in the shafts ranges from about 2:1 to about 1:2.

14. The microneedle patch of embodiment 13, in which the weight ratio of HA to γ-PGA in the shafts is about 1:2.

15. The microneedle patch of any one of embodiments 12 to 14, in which the average molecular weight of the γ-PGA ranges from about 5 kDa to about 800 kDa, about 100 kDa to about 800 kDa, about 100 kDa to about 400 kDa, about 100 kDa to about 150 kDa, about 5 kDa to about 400 kDa, 5 kDa to about 200 kDa, about 5 kDa to about 150 kDa, about 5 kDa to about 100 kDa.

16. The microneedle patch of embodiment 15, in which the average molecular weight of the γ-PGA in the shafts is about 100 kDa.

17. The microneedle patch of any one of embodiments 10 to 16, in which the shafts comprise PVA.

18. The microneedle patch of embodiment 17, in which the average molecular weight of the PVA ranges from about 5 kDa to about 800 kDa, about 100 kDa to about 800 kDa, about 100 kDa to about 400 kDa, about 100 kDa to about 150 kDa, about 5 kDa to about 400 kDa, 5 kDa to about 200 kDa, about 5 kDa to about 150 kDa, about 5 kDa to about 100 kDa.

19. The microneedle patch of any one of embodiments 1 to 18, in which the shafts further comprise one or more cosmetic agents.

20. The microneedle patch of any one of embodiments 1 to 19, in which the tips consist essentially of HA.

21. The microneedle patch of any one of embodiments 1 to 19, in which the tips further comprise one or more cosmetic agents.

22. The microneedle patch of any one of embodiments 1 to 21, wherein the shafts range from about 200 μm to about 1000 μm in height.

23. The microneedle patch of embodiment 22, wherein the shafts are about 400 μm in height.

24. The microneedle patch of any one of embodiments 1 to 23, wherein the tips range from about 200 μm to about 1000 μm in height.

25. The microneedle patch of embodiment 24, wherein the tips are about 380 μm in height.

26. The microneedle patch of any one of embodiments 1 to 25, wherein the shafts have a uniform width throughout their length.

27. The microneedle patch of any one of embodiments 1 to 26, wherein the shafts are about 100 μm to about 500 μm wide.

28. The microneedle patch of embodiment 26 or embodiment 27, wherein the shafts are about 180 μm wide.

29. The microneedle patch of any one of embodiments 1 to 28, wherein the ends of the tips connected to the shafts are about 100 μm to about 500 μm wide.

30. The microneedle patch of embodiment 29, wherein the ends of the tips connected to the shafts are about 180 μm wide.

31. The microneedle patch of any one of embodiments 1 to 30, wherein the width of the ends of the tips connected to the shafts is the same as the width of the shafts.

32. The microneedle patch of any one of embodiments 1 to 31, wherein the microneedles are about 400 μm to about 1200 μm, about 500 μm to about 1000 μm, or about 600 μm to about 800 μm in height.

33. The microneedle patch of any one of embodiments 1 to 32, wherein the shafts have a circular or square cross-section.

34. The microneedle patch of any one of embodiments 1 to 33, wherein the tips are cone shaped or square pyramidal shaped.

35. The microneedle patch of any one of embodiments 1 to 34, wherein the ends of the tips connected to the shafts have the same shape as the cross-sectional shape of the shafts.

36. The microneedle patch of any one of embodiments 1 to 35, wherein the substrate comprises γ-PGA, polyvinylpyrrolidone, polyvinyl alcohol, starch, gelatin, collagen, or a combination thereof.

37. The microneedle patch of embodiment 36, in which the substrate comprises γ-PGA.

38. The microneedle patch of any one of embodiments 1 to 37, comprising about 10 to about 500, about 100 to about 200, about 200 to about 300, about 300 to about 400, about 400 to about 500, about 10 to about 100, about 20 to about 100, about 30 to about 100, about 40 to about 100, or about 50 to about 100 microneedles per square centimeter of substrate.

39. The microneedle patch of embodiment 38, comprising about 80 microneedles per square centimeter of substrate.

40. The microneedle patch of any one of embodiments 1 to 39, further comprising a second substrate material, optionally selected from single-sided tape, double-sided tape, and fabric.

41. A method for delivering hyaluronic acid to the skin of a subject, comprising applying the microneedle patch of any one of embodiments 1 to 40 to the subject's skin.

42. The method of embodiment 41, further comprising removing the microneedle patch after more than 50%, more than 60%, more than 70%, more than 80%, more than 90%, more than 90%, more than 95%, more than 96%, more than 97%, more than 98%, more than 99% or 100% of the tips have separated from the shafts.

43. The method of embodiment 41, further comprising removing the microneedle patch after about 1 to about 15 minutes, about 1 minute to about 10 minutes, about 1 minute to about 5 minutes, about 2 minutes to about 15 minutes, about 2 minutes to about 10 minutes, about 2 minutes to about 5 minutes, about 3 minutes to about 15 minutes, about 3 minutes to about 10 minutes, about 3 minutes to about 5 minutes, about 4 minutes to about 15 minutes, about 4 minutes to about 10 minutes, about 4 minutes to about 5 minutes, about 5 minutes to about 15 minutes, or about 5 minutes to about 10 minutes.

44. The method of embodiment 43, in which the microneedle patch is removed after about 3 minutes, about 4 minutes, about 5 minutes, about 6 minutes, about 7 minutes, about 8 minutes, about 9 minutes, or about 10 minutes.

45. The method of any one of embodiments 41 to 44, comprising applying the microneedle patch one or more times.

46. The method of embodiment 45, comprising applying the microneedle patch more than one time, optionally at regular intervals.

47. The method of embodiment 46, comprising applying the microneedle patch daily, twice weekly, three times weekly, weekly, monthly, or semi-annually, or annually.

48. The method of embodiment 46 or embodiment 47, comprising continuing to apply the microneedle patch at a regular interval for at least 1 week, at least 4 weeks, at least 8 weeks, at least 12 weeks, at least 1 month, at least 6 months, at least 1 year, or indefinitely.

49. The method of any one of embodiments 41 to 48, in which the skin has a wrinkle, a fine line, a scar, or a combination thereof.

50. The method of embodiment 49, in which the application reduces the appearance of the wrinkle, the fine line, the scar, or the combination thereof.

51. The method of any one of embodiments 41 to 50, in which the application increases the moisture level of the skin, reduces the appearance of pores, increases skin volume, smoothes skin texture, improves skin elasticity, improves skin strength, or a combination thereof.

52. A kit comprising one or more microneedle patches according to any one of embodiments 1 to 40 and one or more of a container, a cleaning agent, a microneedle applicator device, a lotion, a cream, an ointment, and instructions for using the one or more microneedle patches.

While various specific embodiments have been illustrated and described, it will be appreciated that various changes can be made without departing from the spirit and scope of the disclosure(s).

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A microneedle patch for delivering hyaluronic acid (“HA”) to skin, comprising a substrate and a plurality of microneedles immobilized on the substrate, wherein the microneedles comprise:
(a) water dissolvable shafts; and
(b) tips comprising HA;
wherein when the patch is applied to skin the shafts dissolve faster than the tips dissolve.

2. The microneedle patch of claim 1, wherein:
(a) when the patch is applied to skin the shafts dissolve in about 1 minute to about 15 minutes, about 1 minute to about 10 minutes, about 1 minute to about 5 minutes, about 2 minutes to 10 minutes, about 2 minutes to about 5 minutes, about 3 minutes to about 10 minutes, or about 3 minutes to about 5 minutes; or
(b) when the patch is applied to skin the shafts dissolve in about 1 minute, about 2 minutes, about 3 minutes, about 4 minutes, about 5 minutes, about 6 minutes, about 7 minutes, about 8 minutes, about 9 minutes, or about 10 minutes.

3. The microneedle patch of claim 1, wherein the shafts comprise HA, optionally wherein:
(a) the HA in the tips has an average molecular weight that is higher than the average molecular weight of the HA in the shafts;
(b) the average molecular weight of the HA in the shafts ranges from about 5 kDa to about 100 kDa, about 5 kDa to about 50 kDa, about 5 kDa to about 25 kDa, or about 5 kDa to about 10 kDa; or
(c) the average molecular weight of HA in the shafts is about 7 kDa.

4. The microneedle patch of claim 1, wherein the average molecular weight of the HA in the tips ranges from about 5 kDa to about 4 MDa, about 100 kdDa to about 1 MDa, about 100 kDa to about 800 kDa, about 100 kDa to about 600 kDa, about 100 kDa to about 500 kDa, or about 200 kDa to about 500 kDa.

5. The microneedle patch of claim 1, wherein the shafts comprise a polymer that is not HA, optionally wherein:
(a) the polymer comprises gamma-polyglutamic acid (“γ-PGA”), polyvinylpyrrolidone (“PVP”), poly(vinyl alcohol) (“PVA”), starch, gelatin, collagen, or a combination thereof;
(b) the shafts comprise γ-PGA; or
(c) the shafts comprise HA and γ-PGA and the weight ratio of HA to γ-PGA in the shafts ranges from about 2:1 to about 1:2.

6. The microneedle patch of claim Error! Reference source not found., wherein the shafts comprise (a) γ-PGA or (b) HA and γ-PGA, and the average molecular weight of the γ-PGA ranges from about 5 kDa to about 800 kDa, about 100 kDa to about 800 kDa, about 100 kDa to about 400 kDa, about 100 kDa to about 150 kDa, about 5 kDa to about 400 kDa, 5 kDa to about 200 kDa, about 5 kDa to about 150 kDa, about 5 kDa to about 100 kDa.

7. The microneedle patch of claim 5, in which the shafts comprise PVA, optionally wherein the average molecular weight of the PVA ranges from about 5 kDa to about 800 kDa, about 100 kDa to about 800 kDa, about 100 kDa to about 400 kDa, about 100 kDa to about 150 kDa, about 5 kDa to about 400 kDa, 5 kDa to about 200 kDa, about 5 kDa to about 150 kDa, about 5 kDa to about 100 kDa.

8. The microneedle patch of claim 1, in which the shafts further comprise one or more cosmetic agents and/or the tips further comprise one or more cosmetic agents

9. The microneedle patch of claim 1, in which the tips consist essentially of HA.

10. The microneedle patch of claim 1, wherein:
(a) the shafts range from about 200 μm to about 1000 μm in height, optionally wherein the shafts are about 400 μm in height;
(b) the tips range from about 200 μm to about 1000 μm in height, optionally wherein the tips are about 380 μm in height;
(c) the shafts have a uniform width throughout their length;
(d) the shafts are about 100 μm to about 500 μm wide, optionally wherein the shafts are about 180 μm wide;
(e) the ends of the tips connected to the shafts are about 100 μm to about 500 μm wide, optionally wherein the ends of the tips connected to the shafts are about 180 μm wide;
(f) the width of the ends of the tips connected to the shafts is the same as the width of the shafts;
(g) the microneedles are about 400 μm to about 1200 μm, about 500 μm to about 1000 μm, or about 600 μm to about 800 μm in height;
(h) the shafts have a circular or square cross-section;
(i) the tips are cone shaped or square pyramidal shaped;
(j) the ends of the tips connected to the shafts have the same shape as the cross-sectional shape of the shafts; or
(k) any combination of (a)-(j).

11. The microneedle patch of claim 1, wherein the substrate comprises γ-PGA, polyvinylpyrrolidone, polyvinyl alcohol, starch, gelatin, collagen, or a combination thereof.

12. The microneedle patch of claim 1, comprising about 10 to about 500, about 100 to about 200, about 200 to about 300, about 300 to about 400, about 400 to about 500, about 10 to about 100, about 20 to about 100, about 30 to about 100, about 40 to about 100, or about 50 to about 100 microneedles per square centimeter of substrate.

13. The microneedle patch of claim 1, further comprising a second substrate material, optionally selected from single-sided tape, double-sided tape, and fabric.

14. A method for delivering hyaluronic acid to the skin of a subject, comprising applying the microneedle patch of claim 1 to the subject's skin.

15. The method of claim 14, further comprising:
(a) removing the microneedle patch after more than 50%, more than 60%, more than 70%, more than 80%, more than 90%, more than 90%, more than 95%, more than 96%, more than 97%, more than 98%, more than 99% or 100% of the tips have separated from the shafts; or
(b) removing the microneedle patch after about 1 to about 15 minutes, about 1 minute to about 10 minutes, about 1 minute to about 5 minutes, about 2 minutes to about 15 minutes, about 2 minutes to about 10 minutes, about 2 minutes to about 5 minutes, about 3 minutes to about 15 minutes, about 3 minutes to about 10 minutes, about 3 minutes to about 5 minutes, about 4 minutes to about 15 minutes, about 4 minutes to about 10 minutes, about 4 minutes to about 5 minutes, about 5 minutes to about 15 minutes, or about 5 minutes to about 10 minutes.

16. The method of claim 14, comprising:
(a) applying the microneedle patch one or more times;
(b) applying the microneedle patch more than one time, optionally at regular intervals;
(c) applying the microneedle patch daily, twice weekly, three times weekly, weekly, monthly, or semi-annually, or annually; or
(d) applying the microneedle patch as in (b) or (c) and continuing to apply the microneedle patch at a regular interval for at least 1 week, at least 4 weeks, at least 8 weeks, at least 12 weeks, at least 1 month, at least 6 months, at least 1 year, or indefinitely.

17. The method of claim 14, in which the skin has a wrinkle, a fine line, a scar, or a combination thereof.

18. The method of claim 17, in which the application reduces the appearance of the wrinkle, the fine line, the scar, or the combination thereof.

19. The method of claim 14, in which the application increases the moisture level of the skin, reduces the appearance of pores, increases skin volume, smoothes skin texture, improves skin elasticity, improves skin strength, or a combination thereof.

20. A kit comprising one or more microneedle patches according to claim 1 and one or more of a container, a cleaning agent, a microneedle applicator device, a lotion, a cream, an ointment, and instructions for using the one or more microneedle patches.
